(19) United States
(12) Patent Application Publication
(10) Pub. No.: US 2016/0361019 A1
(43) Pub. Date: Dec. 15, 2016

(54) DEVICE AND METHOD FOR VIRTUAL ANGIOGRAPHY

(71) Applicant: Sunnybrook Research Institute, Toronto (CA)

(72) Inventors: Eugene Crystal, Toronto (CA); Nomand Robert, Toronto (CA); Benu Sethi, Toronto (CA); George G. Polack, Toronto (CA)

(73) Assignee: Sunnybrook Research Institute, Toronto (CA)

(21) Appl. No.: 15/120,891

(22) PCT Filed: Feb. 26, 2015
(86) PCT No.: PCT/CA2015/050141

§ 371 Date: Aug. 23, 2016

Related U.S. Application Data
(60) Provisional application No. 61/944,883, filed on Feb. 26, 2014.

(51) Int. Cl. A61B 5/00; A61B 6/12; A61B 8/12; A61M 25/09; A61B 5/02; A61B 6/00; A61B 5/055; A61M 25/01; A61B 8/08

(52) U.S. Cl. CPC A61B 5/6853; A61M 25/0108; A61B 6/12; A61B 8/12; A61B 8/5223; A61B 5/02007; A61B 6/504; A61B 8/0891; A61B 5/055; A61M 25/09; A61B 6/4441; A61B 6/4078; A61B 6/4085; A61B 6/4208

(57) ABSTRACT

A system and method for performing a medical procedure is provided. The system includes a device having a shaft that generally extends from a proximal end to a distal end along a longitudinal axis. The distal end of the device is configured to identify a topographical profile of a lumen, wherein the topographical profile is determined by traversing the device a distance along the longitudinal axis of the lumen. The device also includes at least one of a plurality of projections movably coupled to the distal end and configured to provide a coupling with the lumen by extending in a direction substantially orthogonal to the longitudinal axis, and at least one radiopaque marker coupled to the distal end and designed to provide a contrast during imaging of the device.

### CROSS-REFERENCE TO RELATED APPLICATIONS

This application claims the benefit of U.S. Provisional Patent Application Ser. No. 61/944,883, filed on Feb. 26, 2014, and entitled “DEVICE AND METHOD FOR VIRTUAL ANGIOGRAPHY.”

### BACKGROUND OF THE INVENTION

The field of the invention is systems, devices and methods for medical imaging. More particularly, the invention relates to systems, devices and methods for visualizing the inside of vessels and organs of a body using an interventional medical device, such as a catheter.

Contrast angiography continues to be the gold standard for assessment of cardiac and vascular diseases. A patient typically administered contrast agents is likely subjected to substantial amounts of radiation during diagnostic and interventional procedures using fluoroscopic imaging methods. In addition to undesirable effects of radiation, contrast agents can strain kidney functions, and so extra caution needs to be taken for patients suffering from renal disorders, as well as for normal patients, undergoing such procedures.

Rapidly-developing interventional procedures using catheterization have gained popularity as alternatives to surgical procedures for patients at risk for complications or those with severe co-morbidities. For example, trans-catheter aortic valve replacement (TAVR) used to treat aortic valve stenosis has seen more than 50,000 procedures performed since introduced in 2002. In addition, interventional methods addressing other cardiovascular afflictions, such as mitral valve failure, are also seeing increased popularity via catheter techniques.

Accurate positioning of transcatheter delivered tools is crucial to achieve treatment effect and to prevent complications, such as valve malfunctions, flow occlusions, and others.

Therefore, given the above, there exists a need for accurately positioning and identifying a diagnostic or interventional medical device within the three-dimensional space of a subject.

### SUMMARY OF THE INVENTION

The present invention overcomes the aforementioned drawbacks by providing a catheter and method for performing a medical procedure, such as an angiography, without need for administering a contrast agent to a subject.

It is an aspect of the invention to provide a device for obtaining topographical data of an interior surface of a bodily lumen of a subject. The device includes a shaft extending from a proximal end to a distal end along a longitudinal axis, and a profiling assembly coupled to the distal end of the shaft. The profiling assembly is configured to be adjusted between an undeployed configuration and a deployed configuration. The profiling assembly includes a plurality of projections that are movably coupled to and extending away from the distal end of the shaft. In the undeployed configuration, the projections are substantially parallel with the longitudinal axis of the shaft. In the deployed state, the projections are angled away from the longitudinal axis of the shaft and configured to engage an interior surface of a bodily lumen.

It is another aspect of the invention to provide a method for generating a topographical profile of an interior surface of a bodily lumen of a subject using a device and an x-ray imaging system. The device is positioned within a bodily lumen of a subject. The device includes a profiling assembly that is configured to contact an interior surface of the bodily lumen. One or more images of the device are then acquired with an x-ray imaging system. From the one or more images, a position of the device is determined. Topographical data of the interior surface of the bodily lumen is then acquired using the profiling assembly. The topographical data can be acquired from the one or more images of the profiling assembly. In some embodiments, the profiling assembly includes one or more ultrasound transducers, and in these embodiments the acquired topographical data can be ultrasound signals. A topographical profile of the interior surface of the bodily lumen is determined from the topographical data and can be reported together with the position of the device.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A and 1B are schematic illustrations showing an undeployed and deployed state of an example device for obtaining topographical data of the interior surface of a bodily lumen in accordance with some embodiments of the present invention.

FIGS. 2A-2D are schematic illustrations showing different configurations of a profiling assembly for obtaining topographical data of the interior surface of a bodily lumen in accordance with some embodiments of the present invention.

FIG. 3 is a flowchart illustrating setting forth steps of a method of operation in accordance with the present invention.

FIG. 4 illustrates an example of a C-arm x-ray imaging system in accordance with the present invention.

### DETAILED DESCRIPTION OF THE INVENTION

Described here are systems and methods for performing non-contrast angiography using a device that is capable of measuring the topographical profile of the interior surface of a bodily lumen, such as a blood vessel, bodily cavity, or interior of an organ. The device can be localized using a single, two-dimensional x-ray image and thus can be quickly imaged during a procedure, and with minimal dose exposure to the subject. Because the device can measure the topographical profile of the interior surface of a bodily lumen without requiring the administration of a contrast agent, the systems and methods of the present invention are capable of providing angiographic information to subjects that are at-risk or otherwise contraindicated for contrast agent use.

The devices and methods of the present invention as described below encompass a technique that can potentially reduce the use of nephrotoxic contrast agents and fluoroscopic radiation required during, for example, a coronary angiography procedure, and may also improve the accuracy of performing valvular or vascular percutaneous interventions. In addition such devices and methods may find use in applications requiring mapping the interior of organs or cavities. For example, the devices and methods may be used to provide mapping of internal surfaces of a colon to identify polyps. Furthermore, such devices and methods may be advantageous for performing interventional procedures, such as ablation or excision of a tissue.

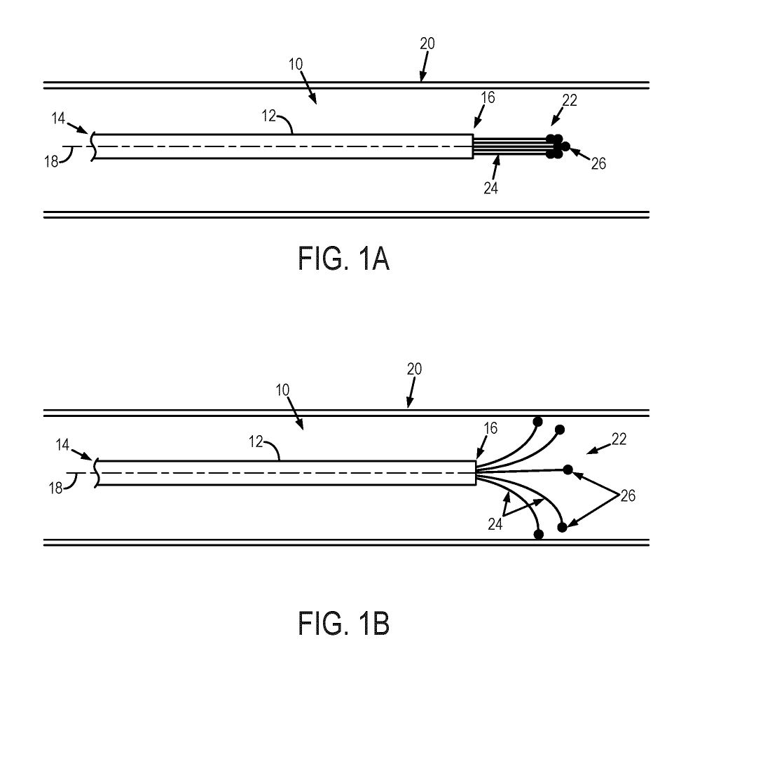

Referring now to FIGS. 1A and 1B, an example device 10 in accordance with the present invention is illustrated. The device 10 may be implemented as a catheter or other suitable diagnostic or interventional device. The device 10 thus generally includes a shaft 12 extending from a proximal end 14 to a distal end 16 along a longitudinal axis 18. The device 10 is preferably configured and dimensioned to be introduced into and traversed within a bodily lumen 20, such as a blood vessel, bodily cavity, or interior of an organ. A profiling assembly 22 for measuring the topological profile of a bodily lumen is coupled to the distal end 16 of the shaft 12.

In some embodiments, the device 10 may be composed in whole or in part of materials that increase the visibility of the device 10 in a particular imaging modality. As an example, the shaft 12 of the device 10 may include one or more markers that are visible in a particular imaging modality. For instance, the markers may be radioopaque markers for x-ray imaging applications. As another example, the shaft 12 of the device 10 may include metallic electrodes or other structures that improve the visibility of the device 10 for a particular imaging modality. For instance, the surface of the shaft 12 may include radioopaque metallic electrodes for x-ray imaging applications.

Generally, the profiling assembly 22 is configured to contact the interior surface of the bodily lumen 20 so that topographical data of the surface can be obtained. As will be described below with respect to different embodiments, the contact may be physical contact or may be sensing contact, which is when the profiling assembly 22 is configured to interact with the interior surface of the bodily lumen 20 without direct physical contact. An example of sensing contact includes interacting with the interior surface of the bodily lumen 20 using ultrasound.

The profiling assembly 22 is constructed to minimize occlusion of the bodily lumen 20 when it is in an operable configuration for measuring the topology of the interior surface of the bodily lumen 20. The device 10 may also be configured to facilitate interventional procedures, such as obtaining a biopsy, removing a tissue, placing a stent or other prosthetic, performing an ablation, and so forth.

In some embodiments, such as those illustrated in FIGS. 1A and 1B, the profiling assembly 22 includes a plurality of deployable projections 24 extending outward away from the distal end 16 of the shaft 12. In these embodiments, the projections 24 can be shaped as flexible or rigid stems, bristles or fingers; although, other shapes and forms are also possible. Preferably, markers 26 are coupled to the projections, so as to facilitate imaging of the device 10 and measurement of the topographical profile of the bodily lumen 20. In some embodiments, the markers 26 may be shaped as spherical markers; however, the marker 26 may also be configured to have any suitable geometric shape and thus may be elliptical, cylindrical, linear, and so forth.

In some embodiments, the markers 26 are composed of a radioopaque material so as to make them visible in x-ray imaging applications. In some embodiments, however, it is preferable to track the device 10 using an imaging modality that does not expose the subject to unnecessary dose of ionizing radiation. In these embodiments, the markers 26 may be composed of a material that has an increased image contrast for that imaging modality. For instance, if magnetic resonance imaging (“MRI”) is utilized to track the device 10, the markers 26 may be composed of a material that constitutes a magnetic resonance contrast agent, such as a paramagnetic material. In some configurations, the markers 26 may include different sets of markers that are visible under different imaging or tracking modalities. As an example, the markers 26 may include a first plurality of markers that are radioopaque for x-ray imaging and a second plurality of markers that are paramagnetic for MRI. Thus, alternative imaging modalities may be used to verify new marker positions before x-ray exposure to the subject.

The profiling assembly 22 illustrated in FIGS. 1A and 1B is operable to be adjusted from an undeployed configuration (FIG. 1A) to a deployed configuration (FIG. 1B). In some embodiments, the profiling assembly 22 is adjusted between the undeployed and deployed configurations by manipulating a guidewire coupled on its distal end to the profiling assembly 22. For instance, manipulation of the guidewire may retract or expand the projections 24.

In the undeployed state, the projections 24 may be tightly clustered or configured to minimize occupying the surrounding volume. This undeployed state facilitates traversal through or insertion into the bodily lumen 20, which may allow for a relatively unimpeded delivery of the profiling assembly 22 to a desired site, location, or region within the bodily lumen 20. In the deployed state, the projections 24 generally extend away from the longitudinal axis 18 of the device 10. Preferably, when extended, the projections 24 may generally contact or conform to the interior surface of the bodily lumen 20 with minimal disturbance, obstruction, or occlusion of the bodily lumen 20. For example, the device 10 may be designed to allow blood flow through a blood vessel during use.

In some embodiments, the profiling assembly 22 may include one or more orientation sensors coupled to the projections 24. The orientation sensors can be provided in addition to, or in lieu of, the markers 26. As an example, the orientation sensors can be configured to measure a deflection of the projections 24 as they contact the interior surface of the bodily lumen 20. Using information about the known geometry of the projections 24, this deflection information can be used to measure the topological profile of the interior surface of the bodily lumen 20.

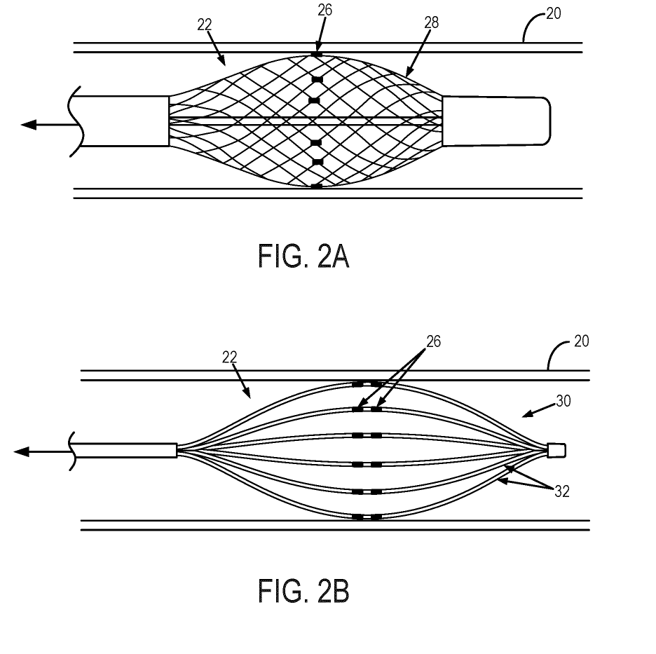

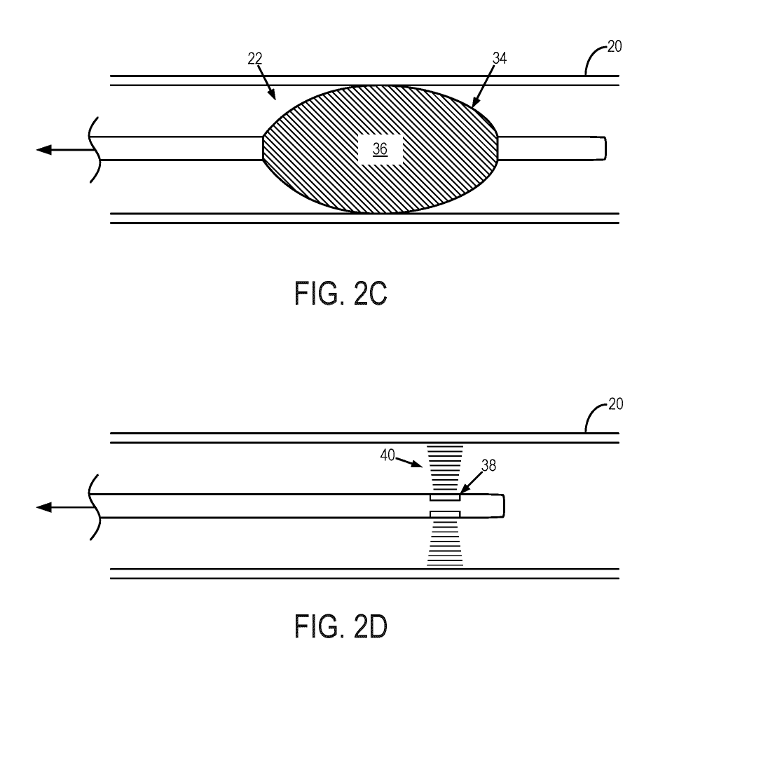

In some embodiments, such as the one illustrated in FIG. 2A, the profiling assembly may include a mesh 28 on which the markers 26 can be coupled. In is deployed state, the mesh 28 expands to come into contact with the interior surface of the bodily lumen 20. Because the markers 26 are coupled to the mesh 28, they too come into contact with the interior surface of the bodily lumen 20. Images of the device 10 in this configuration will depict the spatial extent of the markers 26, thereby providing a measurement of the topographical profile of the bodily lumen 20 at that location.

In some embodiments, such as the one illustrated in FIG. 2B, the profiling assembly may include a basket 30 constructed of a plurality of splines 32 in which the markers 26 may be coupled. In its deployed state, the basket 30 expands to come into contact with the interior surface of the bodily lumen 20. Because the markers 26 are coupled to the basket 30, they too come into contact with the interior surface of the bodily lumen 20. Images of the device 10 in this configuration will depict the spatial extent of the markers 26, thereby providing a measurement of the topographical profile of the bodily lumen 20 at that location.

In some embodiments, such as the one illustrated in FIG. 2C, the profiling assembly may include an inflatable balloon 34 configured to be filled with a contrast agent 36. When the balloon 34 is filled with the contrast agent 36, the balloon 34 is made to come into conformal contact with the interior surface of the bodily lumen 20. Images of the device 10 in this configuration will depict the spatial extent of the contrast-filled balloon 34, thereby providing a measurement of the topographical profile of the bodily lumen 20 at that location. As an example, the contrast agent may be a radioopaque material for x-ray imaging applications. As another example, the contrast agent may be a paramagnetic material for MRI applications.

In some embodiments, such as the one illustrated in FIG. 2D, the profiling assembly includes a plurality of ultrasound transducers or transducer arrays 38. The ultrasound transducers 38 are operable to emit ultrasound 40 and to receive echo signals in response to the emitted ultrasound 40, for example, using an A-mode setting. In this manner, the ultrasound transducers 38 can be used to measure the distance between the profiling assembly 22 and the interior surface of the bodily lumen 20, thereby determining the topological profile of that surface.

In the embodiments that utilize ultrasound transducer 38 to acquire topographical data, the topographical data may include information about the profile of the superficial layers of the bodily lumen 20 and also about the profiles or contours of sub-surface structures. As an example, the topographical data obtained with ultrasound transducers 38 may be capable of discerning the topographical profile of the interior surface of a blood vessel in addition to a measurement of the thickness of plaque in the blood vessel.

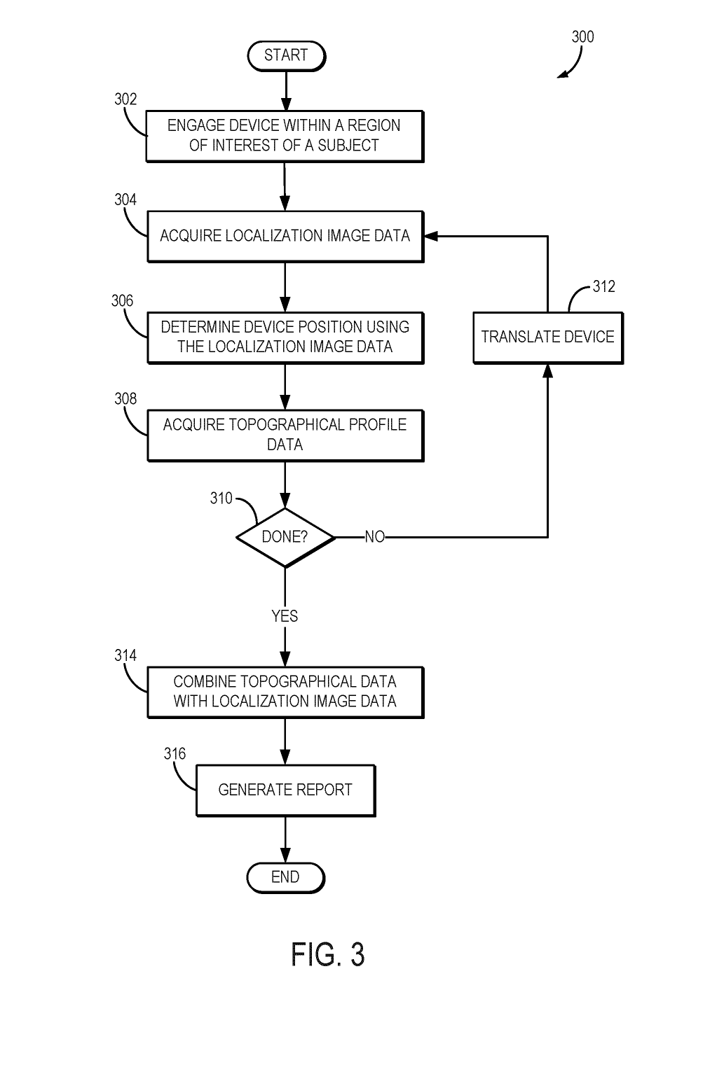

Referring now to FIG. 3, a flowchart 300 setting forth the steps of an example method for performing virtual angiography using a device 10, such as those described above, is illustrated. Generally, the device 10 is translated through the bodily lumen 22 while the profiling assembly 22 is operated to obtain topographical data of the interior surface of the bodily lumen 20. At each position where topographical data is acquired, an image of the device 10 is obtained and used to localize the device 10.

Thus, beginning with process block 302, the device 10 may be positioned and engaged at a specific location or a region-of-interest within the bodily lumen 20. Operation of the device 10 is preferably integrated in or combined with imaging systems to provide localization of the device 10 within the bodily lumen 20. Thus, at process block 304, an image of the device 10 is acquired using an imaging system, such as a x-ray imaging system. Multiple images of the device 10, such as bi-plane images, may be acquired at each position, but it is preferable that only one image be obtained to minimize the radiation dose imparted to the subject.

The position of the device 10 is then determined from the acquired image at process block 306. One possible technique for localizing the device using a single, two-dimensional x-ray image is described in International Patent Application Publication No. WO 2013/106926, which is herein incorporated by reference in its entirety. This localization technique includes determining in real-time the three-dimensional location of an object, such as an interventional medical device or a marker placed thereon, from two-dimensional images acquired with a standard medical imaging system, such as an x-ray imaging system. For example, the position of the device 10 can be determined by iteratively comparing images acquired with the imaging system with template images that depict the object as a two-dimensional synthesized projection image of the object at different positions and orientations. Implementing such an imaging approach allows for real-time three-dimensional localization of the profiling assembly 22 of the device 10 at any location within the bodily lumen 20.

At process block 308, topographical profile data of the interior surface of the bodily lumen 20 is acquired. In some embodiments, the topographical profile data is obtained from an image of the device 10. For example, if the profiling assembly 22 includes markers 26, an image that depicts the profiling assembly can be processed to determine the spatial extent of the interior surface of the bodily lumen 20 at that position. As noted above, when the markers 26 are radioopaque, an x-ray imaging system can be used to obtain the image of the profiling assembly 22. In other examples, however, the markers 26 can be configured to allow imaging of the profiling assembly 22 using other imaging modalities. In these other configurations, the radiation dose imparted to the subject can be further reduced by not requiring measurement of the topographical profile data using x-rays. As another example, if the profiling assembly 22 is configured to determine the spatial orientation and position of the profiling assembly 22, such as via orientation sensors in the device 10, then that information can be used to determine the spatial extent of the interior surface of the bodily lumen 20 at that position. As still another example, if the profiling assembly 22 include one or more ultrasound transducers, the data acquired with those ultrasound transducers can be used to determine the spatial extent of the interior surface of the bodily lumen 20 at that position.

A determination is then made at decision block 310 whether the procedure is completed or whether additional topographical data should be acquired. If more data is to be acquired the device 10 is translated to a new position as indicated at step 312 and steps 304-308 are repeated. The device 10 is thus translated through the bodily lumen 22 while acquiring topographical data of the interior surface of the bodily lumen 22. As will be described below, in some embodiments, translating the device 10 may include directing a robotic system to translate the device 10.

Preferably, the translation of the device 10 is confirmed by acquiring images of the device 10 after the translation in step 312 is performed. As described above, this tracking of the device 10 can be performed using an x-ray imaging system or, in order to reduce the radiation dose imparted to the subject, can also be performed with an alternative imaging modality that does not expose the subject to additional ionizing radiation, such as MRI or ultrasound.

Translation of the device 10 may be operated autonomously or semi-autonomously, using any appropriate or desired systems, for any distance along a longitudinal direction of the bodily lumen 20, either intermittently or continuously. The device 10 may also be configured to retrace a traversed pathway along the bodily lumen 20 and may be operated at any rate of speed, as desired or required. For instance, the translation speed of the device 10 may be dependent on a spatiotemporal resolution of the imaging system. As an example, a slow translation speed may be desired when traversing a region of high interest, such as a stenotic portion of the bodily lumen 20, or any region having high spatial variations.

As an example, translation of the device 10 can be controlled by supplying instructions or input to a robotic system coupled to the proximal end 14 of the device 10 shaft 12. The robotic system is thus configured to translate the device 10 along or within the bodily lumen 20 and optionally to manipulate the profiling assembly 22. Such robotic systems may be in communication with one or more imaging systems and the profiling assembly 22, providing a coordination between image and topographical data acquisition of the device 10. In some configurations, the translation speed of the device 10 may be configured to be generally consistent with a rate of image acquisition, as described. For example, such an acquisition rate may be in the range between 3 and 30 frames or images per second in the instances where an x-ray fluoroscopy system is used to image the device 10. As such, translation speeds may be at least configured to accommodate imaging rates in this range.

The acquisition of image data and topographical data may also be performed in combination with systems and methods intended to minimize confounding effects, such as interference from respiratory or cardiac cycles. To this end, the translation of the device 10 and data acquisition may be appropriately timed with a physiological gating signal.

At process block 314, cross-sectional and localization image data, as described, may be combined. In some envisioned configurations, mapping or information related to tracked reflections, deviations, deflections, or compressions as a function of travel time, or distance traversed in the lumen, vessel or cavity may be combined or registered with localization image data to produce real-time imaging. Such an approach provides information with respect to a lumen, vessel or cavity that advantageously obviates the need for administering a contrast agent and with reduced amounts of radiation exposure. For example, operating the device 100, as described, within a coronary artery may be used to create a virtual angiogram.

Using the acquired topographical data and positional information of the device 10, a topographical profile of the bodily lumen 20 may be generated, as indicated at step 314. The topographical profile conveys information about the contours and curvatures of the bodily lumen, including abnormal narrowings or expansions of the bodily lumen and otherwise abnormal surface features, such as the presence of polyps.

As indicated at step 316, a report that combines the topographical profile and the positional information of the device 10 can then be generated. As an example, a linear representation of the interior surface of the bodily lumen 20 can be generated and displayed. In another example, a three-dimensional rendering of the interior surface of the bodily lumen 20 can be constructed and registered with the acquired images or with another image volume. In yet another example, three-dimensional localized topological data of the interior surface of the lumen spanning a segment of the lumen may be superimposed or fused in a currently acquired, two-dimensional x-ray image. This process can be referred to as “roadmapping,” as described by M. Ayad, et al., in “Real-time image guidance for open vascular neurosurgery using digital angiographic roadmapping,” Neurosurgery, 2007;61(3 Suppl):55-61. In other instances, the three-dimensional localized topographical data may be superimposed or fused into a pre-procedural image, which may be a two-dimensional or three-dimensional image, obtained using CT, MRI, or the like.

In some embodiments, the acquisition of image data and topographical data may be performed in combination with systems that determine whether the device 10 is moving relative to a reference point. As an example, the reference point can be associated with the subject being imaged, such as a point on the vessel into which the device 10 is inserted. As another example, the reference point can be on the imaging system, such as on the x-ray source assembly or x-ray detector assembly. As still another example, the reference point can be associated with a reference device located within the subject, or with a reference device placed on the skin of the subject. The acquisition of an image, or a plurality of images, can then be triggered when a condition for the catheter movement relative to the reference point has been met.

As one example of such triggering, lack of device motion relative to a reference point may trigger image acquisition. For instance, translation of the device 10 along the longitudinal axis of the vessel may trigger image acquisition. The detection of longitudinal motion of the device 10, or absence thereof, can be determined in some instances from a measurement of longitudinal translation of the device 10 outside of the patient, or longitudinal translation of the distal tip of the device 10 by adding an intravascular imaging detector. The detection of motion of the device 10, or absence thereof, relative to a reference device can be determined in some instances by making the device 10 compatible with electromagnetic or impedance based navigation systems.

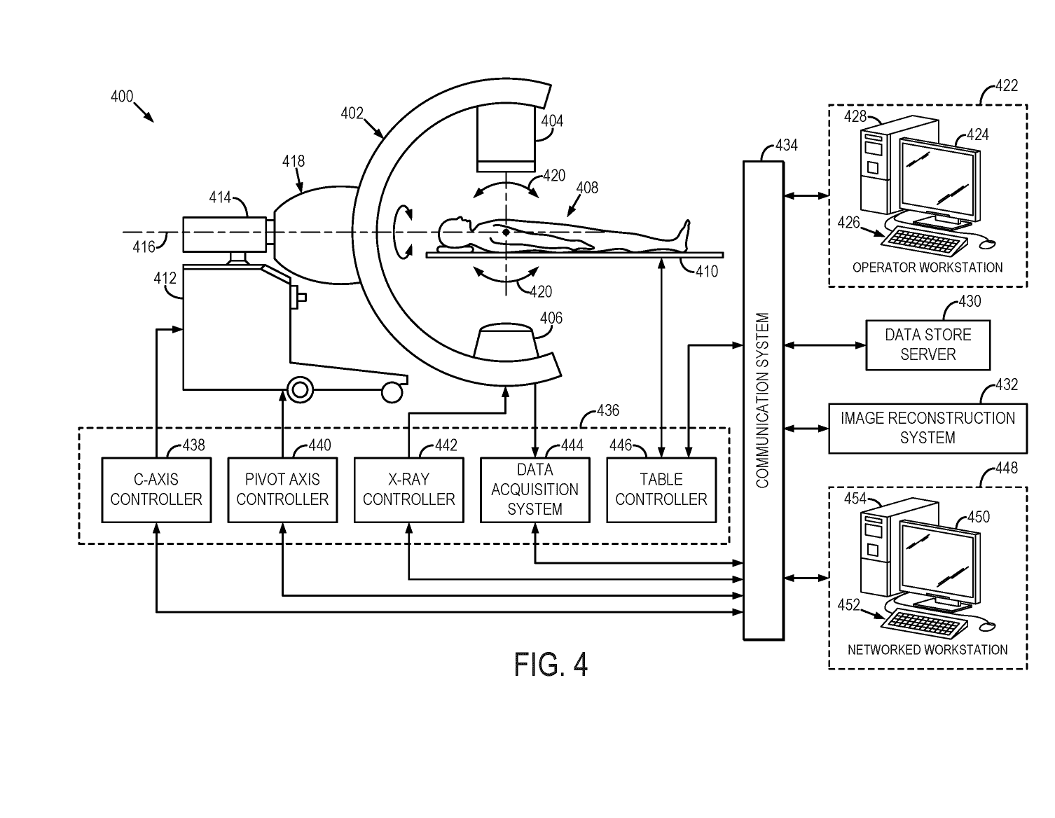

Referring particularly to FIG. 4, an example of a so-called “C-arm” x-ray imaging system 400 is illustrated for use in accordance with some embodiments of the present invention. Such an imaging system is generally designed for use in connection with interventional procedures. The C-arm x-ray imaging system 400 includes a gantry 402 having a C-arm to which an x-ray source assembly 404 is coupled on one end and an x-ray detector array assembly 406 is coupled at its other end. The gantry 402 enables the x-ray source assembly 404 and detector array assembly 406 to be oriented in different positions and angles around a subject 408, such as a medical patient or an object undergoing examination, that is positioned on a table 410. When the subject 408 is a medical patient, this configuration enables a physician access to the subject 408.

The x-ray source assembly 404 includes at least one x-ray source that projects an x-ray beam, which may be a fan-beam or cone-beam of x-rays, towards the x-ray detector array assembly 406 on the opposite side of the gantry 402. The x-ray detector array assembly 406 includes at least one x-ray detector, which may include a number of x-ray detector elements. Examples of x-ray detectors that may be included in the x-ray detector array assembly 406 include flat panel detectors, such as so-called “small flat panel” detectors, in which the detector array panel may be around 20×20 centimeters in size. Such a detector panel allows the coverage of a field-of-view of approximately twelve centimeters.

Together, the x-ray detector elements in the one or more x-ray detectors housed in the x-ray detector array assembly 406 sense the projected x-rays that pass through a subject 408. Each x-ray detector element produces an electrical signal that may represent the intensity of an impinging x-ray beam and, thus, the attenuation of the x-ray beam as it passes through the subject 408. In some configurations, each x-ray detector element is capable of counting the number of x-ray photons that impinge upon the detector. During a scan to acquire x-ray projection data, the gantry 402 and the components mounted thereon rotate about an isocenter of the C-arm x-ray imaging system 400.

The gantry 402 includes a support base 412. A support arm 414 is rotatably fastened to the support base 412 for rotation about a horizontal pivot axis 416. The pivot axis 416 is aligned with the centerline of the table 410 and the support arm 414 extends radially outward from the pivot axis 416 to support a C-arm drive assembly 418 on its outer end. The C-arm gantry 402 is slidably fastened to the drive assembly 418 and is coupled to a drive motor (not shown) that slides the C-arm gantry 402 to revolve it about a C-axis, as indicated by arrows 420. The pivot axis 416 and C-axis are orthogonal and intersect each other at the isocenter of the C-arm x-ray imaging system 400, which is indicated by the black circle and is located above the table 410.

The x-ray source assembly 404 and x-ray detector array assembly 406 extend radially inward to the pivot axis 416 such that the center ray of this x-ray beam passes through the system isocenter. The center ray of the x-ray beam can thus be rotated about the system isocenter around either the pivot axis 416, the C-axis, or both during the acquisition of x-ray attenuation data from a subject 408 placed on the table 410. During a scan, the x-ray source and detector array are rotated about the system isocenter to acquire x-ray attenuation projection data from different angles. By way of example, the detector array is able to acquire thirty projections, or views, per second.

The C-arm x-ray imaging system 400 also includes an operator workstation 422, which typically includes a display 424; one or more input devices 426, such as a keyboard and mouse; and a computer processor 428. The computer processor 428 may include a commercially available programmable machine running a commercially available operating system. The operator workstation 422 provides the operator interface that enables scanning control parameters to be entered into the C-arm x-ray imaging system 400. In general, the operator workstation 422 is in communication with a data store server 430 and an image reconstruction system 432. By way of example, the operator workstation 422, data store sever 430, and image reconstruction system 432 may be connected via a communication system 434, which may include any suitable network connection, whether wired, wireless, or a combination of both. As an example, the communication system 434 may include both proprietary or dedicated networks, as well as open networks, such as the internet.

The operator workstation 422 is also in communication with a control system 436 that controls operation of the C-arm x-ray imaging system 400. The control system 436 generally includes a C-axis controller 438, a pivot axis controller 440, an x-ray controller 442, a data acquisition system (“DAS”) 444, and a table controller 446. The x-ray controller 442 provides power and timing signals to the x-ray source assembly 404, and the table controller 446 is operable to move the table 410 to different positions and orientations within the C-arm x-ray imaging system 400.

The rotation of the gantry 402 to which the x-ray source assembly 404 and the x-ray detector array assembly 406 are coupled is controlled by the C-axis controller 438 and the pivot axis controller 440, which respectively control the rotation of the gantry 402 about the C-axis and the pivot axis 416. In response to motion commands from the operator workstation 422, the C-axis controller 438 and the pivot axis controller 440 provide power to motors in the C-arm x-ray imaging system 400 that produce the rotations about the C-axis and the pivot axis 416, respectively. For example, a program executed by the operator workstation 422 generates motion commands to the C-axis controller 438 and pivot axis controller 440 to move the gantry 402, and thereby the x-ray source assembly 404 and x-ray detector array assembly 406, in a prescribed scan path.

The DAS 444 samples data from the one or more x-ray detectors in the x-ray detector array assembly 406 and converts the data to digital signals for subsequent processing. For instance, digitized x-ray data is communicated from the DAS 444 to the data store server 430. The image reconstruction system 432 then retrieves the x-ray data from the data store server 430 and reconstructs an image therefrom. The image reconstruction system 430 may include a commercially available computer processor, or may be a highly parallel computer architecture, such as a system that includes multiple-core processors and massively parallel, high-density computing devices. Optionally, image reconstruction can also be performed on the processor 428 in the operator workstation 422. Reconstructed images can then be communicated back to the data store server 430 for storage or to the operator workstation 422 to be displayed to the operator or clinician.

The C-arm x-ray imaging system 400 may also include one or more networked workstations 448. By way of example, a networked workstation 448 may include a display 450; one or more input devices 452, such as a keyboard and mouse; and a processor 454. The networked workstation 448 may be located within the same facility as the operator workstation 422, or in a different facility, such as a different healthcare institution or clinic.

The networked workstation 448, whether within the same facility or in a different facility as the operator workstation 422, may gain remote access to the data store server 430, the image reconstruction system 432, or both via the communication system 434. Accordingly, multiple networked workstations 448 may have access to the data store server 430, the image reconstruction system 432, or both. In this manner, x-ray data, reconstructed images, or other data may be exchanged between the data store server 430, the image reconstruction system 432, and the networked workstations 448, such that the data or images may be remotely processed by the networked workstation 448. This data may be exchanged in any suitable format, such as in accordance with the transmission control protocol (“TCP”), the Internet protocol (“IP”), or other known or suitable protocols.

The present invention has been described in terms of one or more preferred embodiments, and it should be appreciated that many equivalents, alternatives, variations, and modifications, aside from those expressly stated, are possible and within the scope of the invention.

1. A device for obtaining topographical data of an interior surface of a bodily lumen of a subject, comprising:
a shaft extending from a proximal end to a distal end along a longitudinal axis;
a profiling assembly coupled to the distal end of the shaft and configured to be adjusted between an undeployed configuration and a deployed configuration, the profiling assembly comprising:
a plurality of projections movably coupled to and extending away from the distal end of the shaft, wherein in the undeployed configuration the projections are substantially parallel with the longitudinal axis of the shaft and in the deployed state the projections are angled away from the longitudinal axis of the shaft and configured to engage an interior surface of a bodily lumen.

2. The device as recited in claim 1, further comprising at least one sensor configured to measure deflections of the plurality of projections as the plurality of projections engage the interior surface of the bodily lumen.

3. The device as recited in claim 1, wherein at least one of the plurality of projections is shaped and dimensioned as a linear stem projection.

4. The device as recited in claim 1, wherein the plurality of projections are rigid projections.

5. The device as recited in claim 1, wherein the plurality of projections are flexible projections.

6. The device as recited in claim 1, further comprising a plurality of radioopaque markers coupled to the profiling assembly such that at least one radioopaque marker is coupled to each of the plurality of projections.

7. The device as recited in claim 6, wherein at least one of the radioopaque markers is at least one of spherical, elliptical, cylindrical, linear, or rectangular.

8. The device as recited in claim 1, wherein the shaft comprises a catheter shaft.

9. A method for generating a topographical profile of an interior surface of a bodily lumen of a subject using a device and an x-ray imaging system, the method comprising:
a) positioning a device within a bodily lumen of a subject, the device including a profiling assembly configured to contact an interior surface of the bodily lumen;
b) acquiring at least one image of the device with an x-ray imaging system;
c) determining a position of the device using the at least one image produced in step b);
d) acquiring topographical data of the interior surface of the bodily lumen using the profiling assembly;
e) determining a topographical profile of the interior surface of the bodily lumen from the topographical data acquired in step d); and
f) generating a report indicative of a topographical profile of the lumen using the determined topographical profile and the determined position of the device.

10. The method as recited in claim 9, wherein acquiring topographical data comprises determining a spatial extent of the profiling assembly from the at least one image of the device acquired in step b).

11. The method as recited in claim 9, wherein the profiling assembly comprises at least one ultrasound transducer and step d) includes acquiring the topographical data by operating the at least one ultrasound transducer to obtain ultrasound signals from the interior surface of the bodily lumen.
